SEQUENCE LISTING (17 sequences)

SEQ ID NO: 1 - protein, 319 aa, Homo sapiens
AQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 2 - protein, 318 aa, Homo sapiens
KGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 3 - protein, 320 aa, Homo sapiens
PPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 4 - protein, 319 aa, Homo sapiens
WVGHRGTVRDYPDFSPSVDAEAIQKAIRGIGTDEKMLISILTERSNAQRQLIVKEYQAAYGKELKDDLKGDLSGHFEHLMVALVTPPAVFDAKQLKKSMKGAGTNEDALIEILTTRTSRQMKDISQAYYTVYKKSLGDDISSETSGDFRKALLTLADGRRDESLKVDEHLAKQDAQILYKAGENRWGTDEDKFTEILCLRSFPQLKLTFDEYRNISQKDIVDSIKGELSGHFEDLLLAIVNCVRNTPAFLAERLHRALKGIGTDEFTLNRIMVSRSEIDLLDIRTEFKKHYGYSLYSAIKSDTSGDYEITLLKICGGDD

SEQ ID NO: 5 - protein, 318 aa, Homo sapiens
ATKGGTVKAASGFNAMEDAQTLRKAMKGLGTDEDAIISVLAYRNTAQRQEIRTAYKSTIGRDLIDDLKSELSGNFEQVIVGMMTPTVLYDVQELRRAMKGAGTDEGCLIEILASRTPEEIRRISQTYQQQYGRSLEDDIRSDTSFMFQRVLVSLSAGGRDEGNYLDDALVRQDAQDLYEAGEKKWGTDEVKFLTVLCSRNRNHLLHVFDEYKRISQKDIEQSIKSETSGSFEDALLAIVKCMRNKSAYFAEKLYKSMKGLGTDDNTLIRVMVSRAEIDMLDIRAHFKRLYGKSLYSFIKGDTSGDYRKVLLVLCGGDD

SEQ ID NO: 6 - protein, 319 aa, Homo sapiens; identical to SEQ ID NO: 1

SEQ ID NO: 7 - protein, 324 aa, Homo sapiens
RVELKGTVRPANDFNPDADAKALRKAMKGLGTDEDTIIDIITHRSNVQRQQIRQTFKSHFGRDLMTDLKSEISGDLARLILGLMMPPAHYDAKQLKKAMEGAGTDEKALIEILATRTNAEIRAINEAYKEDYHKSLEDALSSDTSGHFRRILISLATGHREEGGENLDQAREDAQVAAEILEIADTPSGDKTSLETRFMTILCTRTYPHLRRVFQEFIKMTNYDVEHTIKKEMSGDVRDAFVAIVQSVKNKPLFFADKLYKSMKGAGTDEKTLTRIMVSRSEIDLLNIRREFIEKYDKSLHQAIEGDTSGDFLKALLALCGGED

SEQ ID NO: 8 - protein, 317 aa, Homo sapiens
TQVTQGTIRPAANFDAIRDAEILRKAMKGFGTDEQAIVDVVANRSNDQRQKIKAAFKTSYGKDLIKDLKSELSGNMEELILALFMPPTYYDAWSLRKAMQGAGTQERVLIEILCTRTNQEIREIVRCYQSEFGRDLEKDIRSDTSGHFERLLVSMCQGNRDENQSINHQMAQEDAQRLYQAGEGRLGTDESCFNMILATRSFPQLRATMEAYSRMANRDLLSSVSREFSGYVESGLKTILQCALNRPAFFAERLYYAMKGAGTDDSTLVRIVVTRSEIDLVQIKQMFAQMYQKTLGTMIAGDTSGDYRRLLLAIVGQ

SEQ ID NO: 9 - protein, 320 aa, Homo sapiens
IEQEGVTVKSSSHFNPDPDAETLYKAMKGIGTNEQAIIDVLTKRSNTQRQQIAKSFKAQFGKDLTETLKSELSGKFERLIVALMYPPYRYEAKELHDAMKGLGTKEGVIIEILASRTKNQLREIMKAYEEDYGSSLEEDIQADTSGYLERILVCLLQGSRDDVSSFVDPALALQDAQDLYAAGEKIRGTDEMKFITILCTRSATHLLRVFEEYEKIANKSIEDSIKSETHGSLEEAMLTVVKCTQNLHSYFAERLYYAMKGAGTRDGTLIRNIVSRSEIDLNLIKCHFKKMYGKTLSSMIMEDTSGDYKNALLSLVGSDP

SEQ ID NO: 10 - protein, 318 aa, Homo sapiens
AWGTLGTLRTFLNFSVDKDAQRLLRAITGQGVDRSAIVDVLTNRSREQRQLISRNFQERTQQDLMKSLQAALSGNLERIVMALLQPTAQFDAQELRTALKASDSAVDVAIEILATRTPPQLQECLAVYKHNFQVEAVDGITSETSGILQDLLLALAKGGRDSYSGIIDYNLAEQDVQALQRAEGPSREETWVPVFTQRNPEHLIRVFDQYQRSTGQELEEAVQNRFHGDAQVALLGLASVIKNTPLYFADKLHQALQETEPNYQVLIRILISRCETDLLSIRAEFRKKFGKSLYSSLQDAVKGDCQSALLALCRAEDM

SEQ ID NO: 11 - protein, 321 aa, Homo sapiens
GDYVQGTIFPAPNFNPIMDAQMLGGALQGFDCDKDMLINILTQRCNAQRMMIAEAYQSMYGRDLIGDLREQLSDHFKDVMAGLMYPPPLYDAHELWHAMKGVGTDENCLIEILASRTNGEIFQMREAYCLQYSNNLQEDIYSETSGHFRDTLMNLVQGTREEGYTDPAMAAQDAMVLWEACQQKTGEHKTMLQMILCNKSYQQLRLVFQEFQNISGQDMVDAINECYDGYFQELLVAIVLCVRDKPAYFAYRLYSAIHDFGFHNKTVIRILIARSEIDLLTIRKRYKERYGKSLFHDIRNFASGHYKKALLAICAGDAEDY

SEQ ID NO: 12 - protein, 319 aa, Homo sapiens
QFGSRGTITDAPGFDPLRDAEVLRKAMKGFGTDEQAIIDCLGSRSNKQRQQILLSFKTAYGKDLIKDLKSELSGNFEKTILALMKTPVLFDIYEIKEAIKGVGTDEACLIEILASRSNEHIRELNRAYKAEFKKTLEEAIRSDTSGHFQRLLISLSQGNRDESTNVDMSLAQRDAQELYAAGENRLGTDESKFNAVLCSRSRAHLVAVFNEYQRMTGRDIEKSICREMSGDLEEGMLAVVKCLKNTPAFFAERLNKAMRGAGTKDRTLIRIMVSRSETDLLDIRSEYKRMYGKSLYHDISGDTSGDYRKILLKICGGND

SEQ ID NO: 13 - protein, 314 aa, Homo sapiens
GNRHAKASSPQGFDVDRDAKKLNKACKGMGTNEAAIIEILSGRTSDERQQIKQKYKATYGNRHAKASSPQGFDVDRDAKKLNKACKGMGTNEAAIIEILSGRTSDERQQIKQKYKATYIIAIKEAYQRLFDRSLESDVKGDTSGNLKKILVSLLQANRNEGDDVDKDLAGQDAKDLYDAGEGRWGTDELAFNEVLAKRSYKQLRATFQAYQILIGKDIEEAIEEETSGDLQKAYLTLVRCAQDCEDYFAERLYKSMKGAGTDEETLIRIVVTRAEVDLQGIKAKFQEKYQKSLSDMVRSDTSGDFRKLLVALLH

SEQ ID NO: 14 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 15 - protein, 157 aa, Homo sapiens [sequence omitted]

SEQ ID NO: 16 - protein, 235 aa, Homo sapiens
MSTESMIRDVELAEEALPQKMGGFQNSRRCLCLSLFSFLLVAGATTLFCLLNFGVIGPQRDEKFPNGLPLISSMAQTLTLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 17 - protein, 157 aa, Homo sapiens
MLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

(19) United States
(12) Patent Application Publication
(10) Pub. No.: US 2018/0154012 A1
(43) Pub. Date: Jun. 7, 2018

(54) CANCER IMMUNOTHERAPEUTIC

(71) Applicant: Rubicon Biotechnology LLC, Lake Forest, CA (US)

(72) Inventors: Missag Parseghian, Hollywood, CA (US); Richard Richieri, Orange, CA (US); Glenn Reynolds, Great Falls, MT (US)

(21) Appl. No.: 15/571,725

(22) PCT Filed: May 5, 2016
(86) PCT No.: PCT/US16/31049

§ 371 Date: Nov. 3, 2017

Related U.S. Application Data
(60) Provisional application No. 62/157,395, filed on May 5, 2015.

(51) Int. Cl. A61K 47/68; C07K 14/47; C07K 14/525; A61P 35/00

(52) U.S. Cl. CPC A61K 47/6811; C07K 14/4721; G01N 33/68; A61P 35/00; C07K 2319/30; C07K 14/525

(57) ABSTRACT

The invention relates to immunotherapeutic compositions and methods for treating specifically solid tumours and cells of the tumour microenvironment.

### RELATED APPLICATIONS

The present invention claims the benefit of priority of U.S. Provisional Application No. 62/157,395, filed May 5, 2015, the contents of which is hereby incorporated by reference in its entirety.

### INCORPORATION BY REFERENCE

The content of the following submission on ASCII text file is incorporated herein by reference in its entirety: a computer readable form (CRF) of the Sequence Listing (file name: 48516-501001WO_SEQLIST.txt, date recorded: May 5, 2016, size: 45,056 bytes).

### FIELD

The invention relates to immunotherapeutic compositions and methods for treating cancer, specifically solid tumours and cells of the tumour microenvironment.

### BACKGROUND

A strategy for generating new anti-cancer therapeutics involves identifying differences between normal, healthy cells and tumour cells and cells of the tumour microenvironment. The aim is to exploit those differences to target therapeutics to tumour cells and cells of the tumour microenvironment, leaving the normal healthy cells unaffected.

Differences have been identified between normal, healthy cells and tumour cells and cells of the tumour microenvironment in the composition of phospholipids in plasma membranes. Certain phospholipids which are largely absent from the outer leaflet of the plasma membrane of normal, healthy cells become exposed on the outer leaflet of tumour cells and other cells of the tumour microenvironment.

An example of a phospholipid which is usually absent from the outer plasma membrane leaflet is phosphatidyl serine (PS). However, in tumour cells and cells of the tumour microenvironment PS is surface exposed. PS surface exposure on tumour cells suppresses the body's immune response to those cells. This effect of PS is accomplished via signaling to macrophages to engulf the cells where PS is located on the outer surface of the plasma membrane. The macrophage engulf a cell, rather than release inflammatory cytokines and other signalling agents, prevents a large scale immune response. Hence, this exposure inhibits inflammatory cytokine signalling from macrophages and provides one avenue by which the tumor microenvironment promotes immunosuppression.

Annexins are proteins which bind phospholipids. Annexin A5 has very high binding affinity for PS, thereby offering a potential tumour targeting molecule. A molecule such as the Fc fragment of an Ig linked to an annexin A5 could offer a conjugate which masks PS and so masks its immunosuppressive effect and provides an immunostimulatory effect. However, annexin A5 is rapidly internalised after binding to surface exposed PS limiting the effect of such a therapeutic strategy.

### SUMMARY OF THE INVENTION

The present invention relates to an anti-cancer conjugate comprising an annexin which masks an immunosuppressive effect of a surface exposed phospholipid on a tumour cell or a cell of the tumour microenvironment, causes immunostimulation at the tumour site and persists at the outer surface of a cell at the tumour site to concentrate and/or prolong these effects in comparison with previously proposed anti-cancer therapeutics involving annexin. Furthermore, a single conjugate of the invention can provide the masking of the immunosuppressive effect of a phospholipid and immunostimulation.

The invention provides a conjugate comprising a non-internalising annexin capable of binding to at least one phospholipid and an immunostimulatory agent.

The invention further provides a polypeptide or nucleotide sequence encoding the conjugate of the invention.

The invention also provides the conjugate, polypeptide or nucleotide sequence disclosed herein for use in a method of medical treatment.

The invention further provides a method of treating cancer comprising administering to a patient in need thereof an effective amount of a conjugate comprising a non-internalising annexin capable of binding to at least one phospholipid and an immunostimulatory agent. The invention also provides the conjugate, polypeptide or nucleotide sequence described herein for use in a method of treating cancer.

Advantageously, the phospholipid to which the conjugate of the invention can bind is selected from phosphatidylserine (PS), phosphatidyl ethanolamine (PE), phosphatidyl inositol (PI) and its phosphoinositide derivatives (PIP, PIP2, PIP3), and preferably PS.

Preferably, the non-internalising annexin is annexin A5. Suitably, one or more amino acids selected from polar amino acids His, Glu, Gln, Asp, Asn, Arg and Lys in the helices IA, ID, IIA, IID, IIIC, IIID and IVE and in the stretches connecting helices IC and ID, IIE and IIIA, IIIC and IIID, IIID and IIIE, and IVA and IVB of annexin are replaced by non-polar amino acids to provide non-internalising annexin. Preferably, the non-internalising annexin comprises at least a portion of SEQ ID NO:1 for annexin A5 or the corresponding sequences for other annexins. Optionally, the one or more amino acids are located at positions 16-29, 59-74, 88-102, 135-145, 156-169, 202-231, 259-266 and 305-317 of SEQ ID NO:1 for annexin A5, or the corresponding sequences for other annexins.

Preferably, the non-internalising annexin comprises SEQ ID NO:1 for annexin A5 or the corresponding sequences for other annexins, wherein one or more amino acids selected from Glu, Gln, Asp, Asn, Arg, Lys and His at positions 16-29, 59-74, 88-102, 135-145, 156-169, 202-231, 259-266 and 305-317, or the corresponding sequences for other annexins, are replaced by Gly, Ala, Val, Ile, Leu, Ser, Thr, Met, Pro, Phe, or Tyr. Suitably, at least two of said polar amino acids are replaced by non-polar amino acids, preferably at least 3, 4, 5 or 6 of said polar amino acids are replaced by non-polar amino acids.

Suitably, the annexin is capable of binding to the phospholipid with a dissociation constant of about 10⁻⁶ M or less, preferably 10⁻⁷ M or less, more preferably 10⁻⁸ M or less, even more preferably 10⁻⁹ M or less.

Preferably, the immunostimulatory agent promotes an inflammatory response.

The immunostimulatory agent may be a nanoparticle such as a fullerene, carbon nanotube, dendrimer or other synthetically constructed supramolecular structure. For example, the nanoparticle may be a C60 nanoparticle, such as polyhydroxy-C60 (poly-C60) or N-ethyl-polyamino-C60 (nepo-C60).

Suitably, the immunostimulatory agent is selected from TNFα, IL1α, IL1β, IL2, IL4, IL6, IL8, IL10, IL12, IL15, IL17A, IFNγ, GM-CSF (CSF2), M-CSF (CSF1), G-CSF (CSF 3), or other applicable cytokines or chemokines, an immunoglobulin, and fragments, monomers, multimers, variants, muteins, post-translationally modified versions thereof and mixtures thereof.

Preferably, the immunostimulatory agent is a fragment crystallizable (Fc) region of an immunoglobulin, monomer or fragment thereof, more preferably IgG, even more preferably human IgG1 or IgG3.

Preferably, the immunostimulatory agent is TNF-α.

Advantageously, the non-internalising annexin is linked to the immunostimulatory agent via a linker. Preferably, the non-internalising annexin is linked to the immunostimulatory agent via the N- or C-terminus, preferably via the N-terminus. Suitably, the conjugate comprises a plurality of immunostimulatory agents and optionally a plurality of linkers.

Alternately, the non-internalizing annexin is linked to a liposome containing one or more of the immunostimulatory agents.

Optionally cancer treatable by a conjugate of the present invention is a solid tumour. The tumour may be selected from breast, triple negative breast, ovarian, prostate, castrate-resistant prostate, pancreatic, bladder, bone, head and neck, lung, liver, thyroid, esophageal, stomach, intestinal, brain, glioblastoma.

### BRIEF DESCRIPTION OF THE DRAWINGS

FIG. 1 depicts the amino acid sequence for wild-type annexin A5 (SEQ ID NO: 1).

FIG. 2 depicts an amino acid sequence alignment between human annexins A1, A2, A3, A4, A5, A6, A7, A8, A9, A10, A11 and A13 (SEQ ID NOS: 2-13). Amino acid positions 16-29, 59-74, 88-102, 135-145, 156-169, 202-231, 259-266, and 305-317 of annexin A5 and corresponding positions of other annexins are underlined in FIG. 1. One or more of these amino acids may be replaced to provide a non-internalising annexin variant. Amino acids located at the concave side of the annexin molecule are represented by italic amino acid symbols in FIG. 1. Amino acid positions 1-15, 46-58, 86-87, 118-134, 170, 245-248 and 280-294 of annexin A5 and the corresponding stretches in other annexins are the bold italic positions in FIG. 1.

FIG. 3 depicts a conjugate comprising a non-internalising annexin A5 and IgG Fc.

FIG. 4 depicts a conjugate comprising a non-internalising annexin A5 and TNF-α.

FIG. 5 depicts amino acid sequences for TNF-α SEQ ID NOS: 14-17.

### DETAILED DESCRIPTION

The invention provides a conjugate comprising a non-internalising annexin capable of binding to at least one phospholipid and an immunostimulatory agent.

As used herein, a “conjugate” is a molecule comprising at least two parts associated such that the parts of the molecule remains associated if transported to a target. Conjugates include fusion proteins linked to each other via their polypeptide structure, through genetic expression of a DNA molecule encoding these proteins, directly synthesised proteins and coupled proteins in which pre-formed sequences are associated by cross-linking agents or associations, such as aggregates of the parts of the molecule.

An “annexin” is a protein characterised by its ability to bind phospholipid, particularly anionic phospholipid, in a calcium dependent manner. Annexins are also characterised by a 70 amino acid repeat sequence called an annexin repeat. The basic structure of an annexin comprises two major domains. The first is located at the COOH terminal and is called the “core” region. The second is located at the NH₂ terminal and is called the “head” region. The core region consists of an alpha helical disk. The convex side of this disk has type 2 calcium-binding sites important for allowing interaction with the phospholipids at the plasma membrane.

As used herein, “non-internalising” in the context of a non-internalising annexin means an annexin that, once bound to phospholipid, will remain on the cellular surface for a longer period of time compared to a wild-type annexin. A non-internalising annexin may have reduced ability to form an annexin trimer, or reduced ability for an annexin trimer to form a lattice with other annexin trimers or both. In the case of annexin A5, a two-dimensional lattice is not formed that would otherwise bend the plasma membrane nanomechanically to elicit budding and endocytic vesicle formation leading to pinocytosis. A non-internalising annexin typically has one or more amino acids replaced in helices IA, ID, IIA, IID, IIIC, IIID and IVE and in the stretches connecting helices IC and ID, IIE and IIIA, IIIC and IIID, IIID and IIIE, and IVA and IVB.

By “immunostimulatory agent” is meant an agent that stimulates the immune system by inducing activation or increasing activity of any of its components. Preferably, the agent promotes an inflammatory response.

As used herein, “post-translationally modified” or a “post-translational modification” includes, but is not limited to, phosphorylation, acetylation, methylation, ubiquitination, sumoylation, hydroxylation, citrullination (deimination), deamidation, oxidation, reduction, or glycosylation. The modification can occur either through biological processes (such as enzymatically in vivo or in vitro) or through synthetic processes (such as a chemical reaction in vitro).

As used herein, “immunoglobulin” (also known as “antibody”) is a protein produced by plasma cells that is used by the immune system to identify and neutralize pathogens such as bacteria and viruses via a specific interaction with an antigen. The protein is typically made of basic structural units—each with two large heavy chains and two small light chains. The fragment crystallisable (or Fc) region of an immunoglobulin is composed of two heavy chains that contribute two or three constant domains depending on the class of the antibody. The Fc region ensures that each antibody generates an appropriate immune response for a given antigen.

By “cancer” is meant a family of diseases that involve abnormal cell growth with the potential to invade or spread to other parts of the body. A tumour is a group of cells that are transformed and grow without normal cell regulation. Reference to treating cancer or treating a tumour can include cells of a tumour mass and cells of the tumour microenvironment, such as tumour vasculature and stromal cells (e.g. fibroblasts and immune cells) for example.

Phospholipids are a class of lipids having a hydrophilic phosphate “head” and two hydrophobic “tails” usually consisting of 2 long fatty acid hydrocarbon chains. Phospholipids are a major component of all cell membranes as they can form lipid bilayers. A lipid bilayer, also known as a phospholipid bilayer, is a sheet of lipids two molecules thick, arranged so that the hydrophilic heads point “out” to the water on either side of the bilayer and the hydrophobic tails point “in” to the core of the bilayer. This arrangement results in two “leaflets” assembled due to the hydrophobic effect.

In many naturally occurring bilayers, the compositions of the inner and outer membrane leaflets are different.

The phospholipids phosphotidyl serine (PS) and phosphotidyl inositol (PI) and its derivatives (PIP, PIP2, PIP3) are largely absent from the surface of healthy mammalian cells under normal conditions with 96% or more found on the inner leaflet of the plasma membrane. PE is mostly in the inner leaflet of resting mammalian cells under normal conditions, with approximately 20% present in the outer leaflet.

Sequestration of PS to the cytosolic leaflet is driven by ATP-hydrolysis and dependent on an aminophospholipid translocase (APLT), whose actions under certain conditions such as apoptosis are countered by a “scramblase” activity that disrupts the membrane asymmetry of PS. Other conditions where PS is detected on the external cell surface include aging erythrocytes, activated platelets and macrophages, and necrotic cells. Disruption of PS asymmetry also appears to be a consequence of hypoxia and oxidative stress as demonstrated in vitro.

Cell surface exposure of PS has several roles, including promoting coagulation in damaged blood vessels and mediating attachment of T-cells to thrombin-activated endothelial cells. It also plays a role in suppressing an auto-immune response against self-antigens by signalling macrophages to engulf cells on which PS is surface exposed while modulating cytokine production.

Clearance of apoptotic cells by phagocytes, termed efferocytosis, plays a pivotal role in normal homeostasis. Efferocytosis is important to promote an anti-inflammatory phenotypic change in macrophages by causing the release of anti-inflammatory cytokines, antiproteinases and growth factors. It has also been shown to reduce proinflammatory mediators such as TNF-alpha. Phosphatidylserine (PS) is involved in one pathway to initiate efferocytosis. Apoptotic cells lose phospholipid asymmetry of the plasma membrane, and PS is exposed on the outer leaflet of the lipid bilayer in the early phase of an apoptotic cell. Therefore, PS acts as signal for efferocytosis. In addition, PS-mediated efferocytosis has proven effects on macrophage function both in vitro and in vivo.

PS exposure on the cell surface is a characteristic of cancer cells, particularly solid tumour cells and cells associated with the tumour vasculature. Conditions of hypoxia and oxidative stress are also common in the tumour microenvironment.

Tumours avoid a full immunological assault by manipulating key “checkpoints” designed to prevent auto-immune disease. Blockading these “checkpoints” has included development and FDA-approval of ipilimumab, an antibody targeting CTLA-4 to inhibit downregulation of T-cell activation. It has also included development of antibodies to disrupt the PD-1/PD-L1 ligand-receptor complex. Yet, these therapies do not localize their activity to the tumours alone and affect the overall immune system.

While the role of surface-exposed PS appears to be key in helping suppress the immune response against dead and dying cells, PS exposure on living tumour cells and their vasculatures provides one avenue by which the tumour microenvironment promotes immunosuppression.

A conjugate according to the invention comprises a non internalising annexin capable of binding to at least one phospholipid. Preferably the phospholipid is not found on the surface of healthy, normal mammalian cells. Preferably the phospholipid is expressed on the surface of cancer cells only, or is expressed in a different amount on the surface of cancer cells compared to healthy, normal mammalian cells.

Anionic phospholipids appear on the surface of cancer cells, particularly tumour cells and cells of the tumour microenvironment and certain aging or apoptotic cells. Anionic phospholipids include phosphatidyl serine (PS), phosphatidic acid (PA), phosphatidyl glycerol (PG), cardiolipin (CL), phosphatidyl inositol (PI) and its phosphoinositide derivatives (PIP, PIP2, PIP3).

Neutral phospholipids include phosphatidyl ethanolamine (PE), phosphatidyl choline (PC) and sphingomyelin (SM).

Aminophospholipids are phospholipids that include within their structure at least a first primary amino group and occur in mammalian cell membranes. PS and PE are examples of aminophospholipids.

In particular, cancer cells can be characterised by cells displaying extracellular phosphatidylserine (PS), phosphatidyl inositol (PI) or its phosphoinositide derivatives (PIP, PIP2, PIP3); or by displaying a different quantity of extracellular phosphatidyl ethanolamine (PE) compared to healthy, normal mammalian cells.

Preferably, the cancer cells can be characterised by cells displaying extracellular anionic aminophospholipid. Preferably, the conjugate comprises a non-internalising annexin capable of binding to PS.

Annexin A5 (Annexin V) is a natural high-affinity PS-binding protein (Kd=100 pM at the physiological ionic strength of 150 mM). It is a 36 kD non-glycosylated protein containing four domains, only one of which (Domain I) binds PS in a Ca2+ ion-dependent manner. Annexin A5 is present as a monomer when in solution, but once bound to PS on the membrane surface, it assembles into trimeric structures; in turn, each trimer interacts with other trimers in its proximity to form a two-dimensional lattice covering the PS-exposed surface. Trimerized annexin binding of PS has multiple roles, including inhibition of PS-triggered coagulation in blood vessels and repair of disrupted plasma membranes. A two-dimensional Annexin A5 lattice will inwardly bend the plasma membrane nanomechanically to elicit budding and endocytic vesicle formation leading to pinocytosis.

Human variants of Annexin A5 that do not internalize upon PS binding have been developed as an apoptosis imaging agent. Mutation of key amino acid residues responsible for salt bridge formation between Annexin A5 Domains I and III result in a variant that still binds PS but does not form a two-dimensional lattice and/or does not elicit inward bending of the plasma membrane. However, conjugation of this annexin to a therapeutic agent has not been contemplated because of predicted toxic side-effects.

Annexin A5 inhibits PS-mediated efferocytosis. This protein interferes with the recognition of apoptotic cells by macrophages, leading to attenuated efferocytosis. Furthermore, mutant annexin A5, which has lost binding affinity to PS, does not affect macrophage efferocytosis. Annexin A5 has been shown to attenuate macrophage efferocytosis in vitro and in vivo, alter the macrophage phenotype toward a more inflammatory one and result in an increase in the amount of inflammatory-associated disease.

A conjugate according to the invention can comprise a non-internalising annexin A1; annexin A10; annexin A11; annexin A13; annexin A2; annexin A3; annexin A4; annexin A5; annexin A6; annexin A7; annexin A8; annexin A8L1; annexin A8L2; or annexin A9. Preferably the annexin is annexin A5.

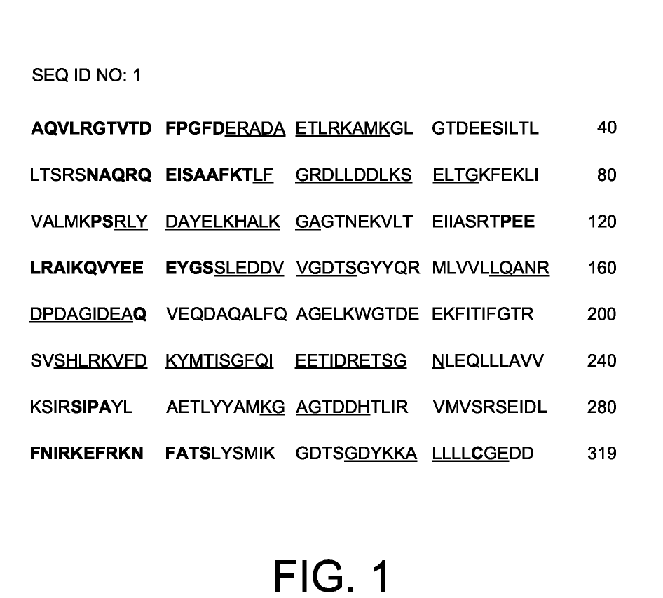

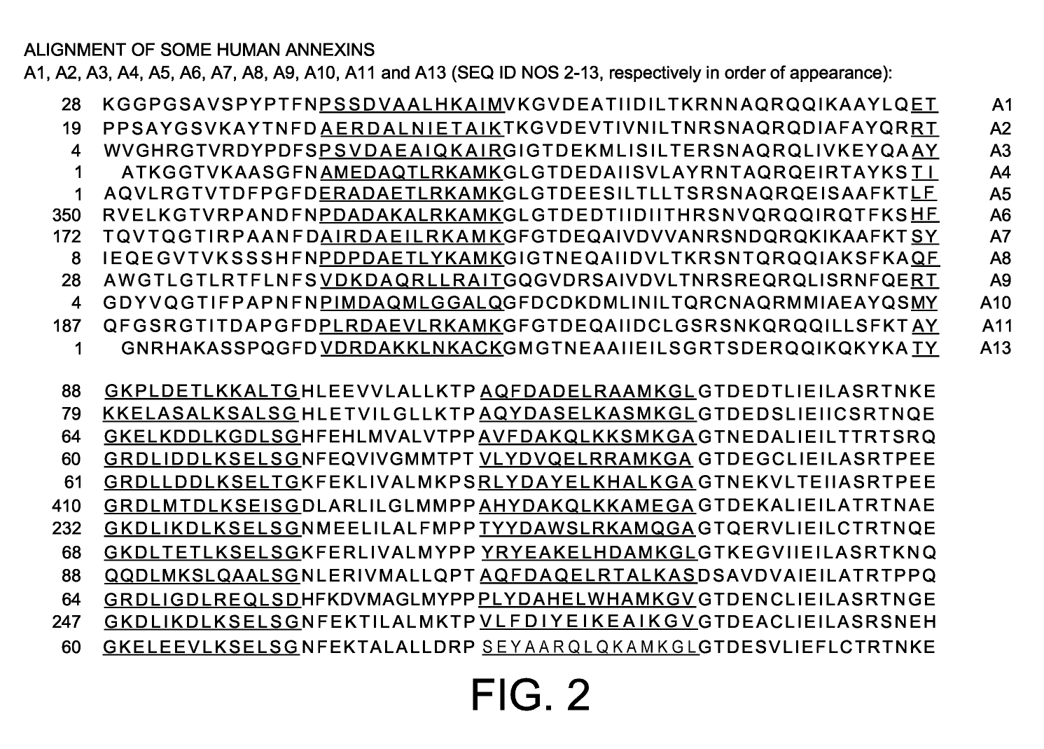

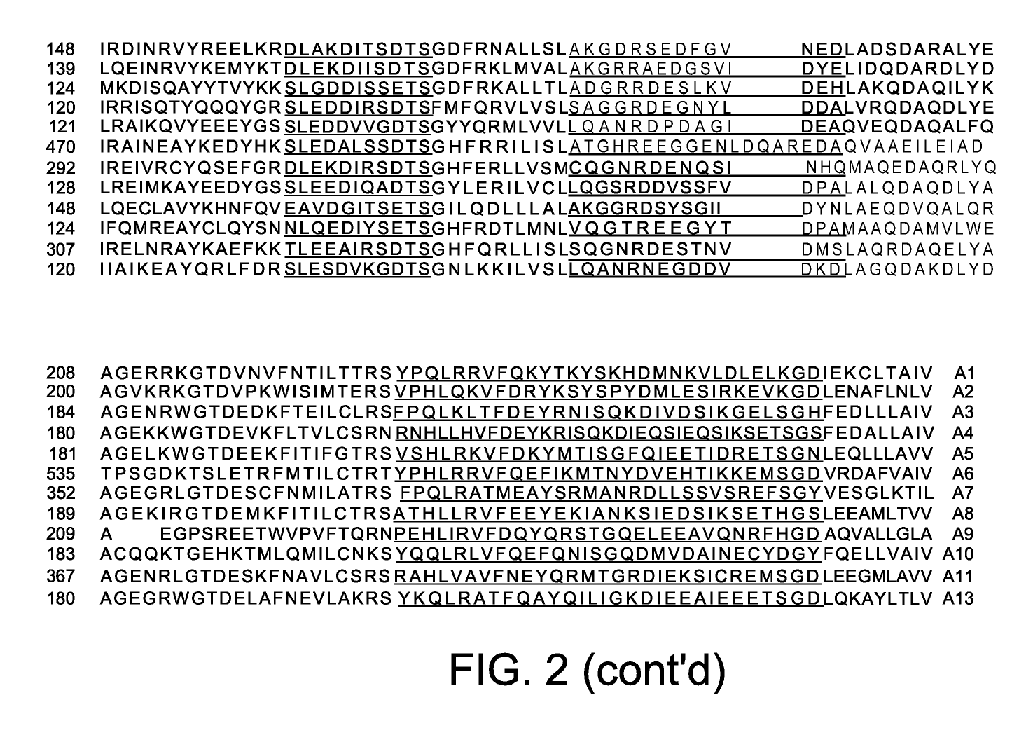

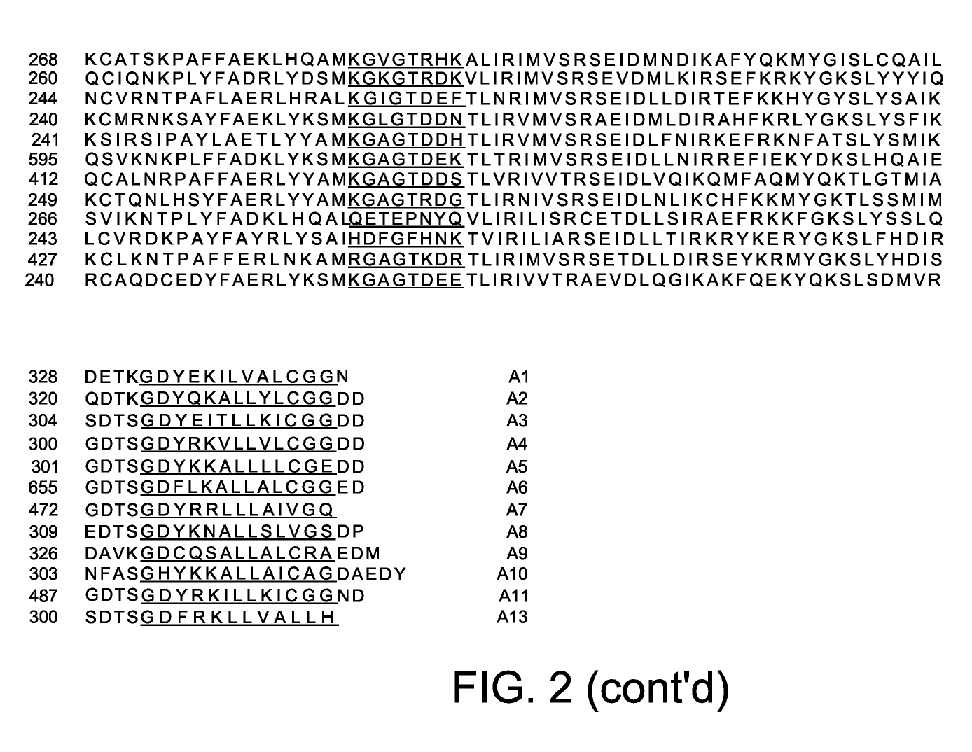

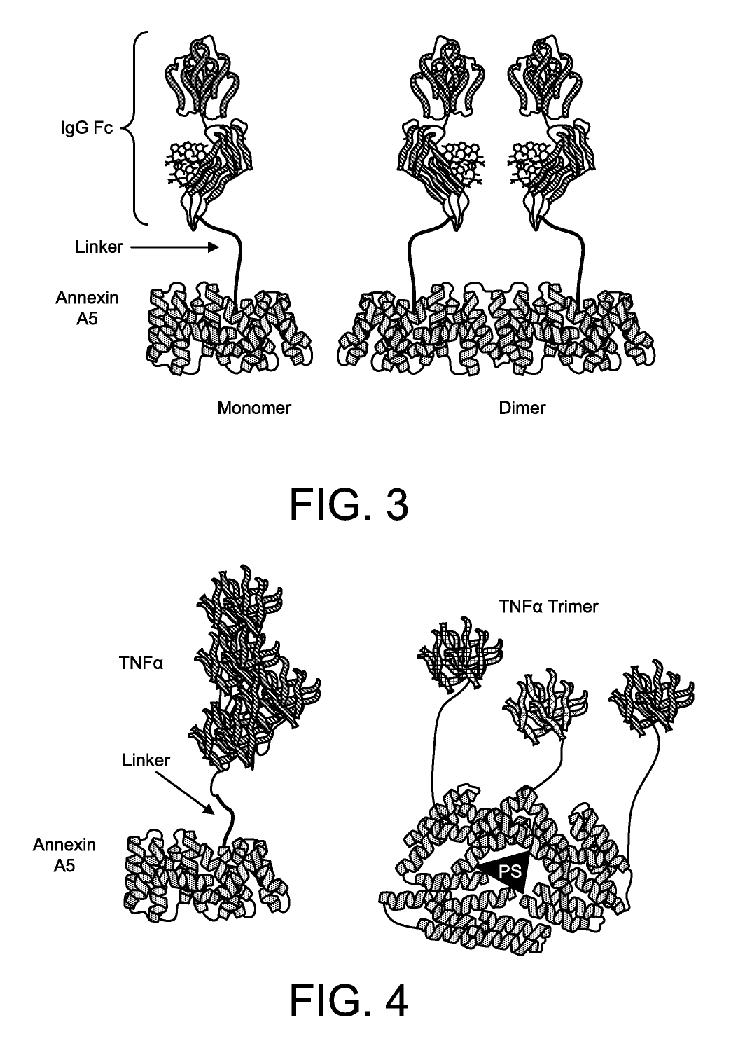

Wild type Annexin A5 can have the amino acid sequence of SEQ ID No. 1 (see FIG. 1). The present invention embraces conjugates comprising non-internalising variants of the annexins listed above. Reference is made to the amino acid sequence and the positions of annexin A5, but what applies to annexin A5 also applies to the other annexins, especially human annexins, by choosing the corresponding position found with the alignment of any annexins (see alignment of human annexins A1 to A11 and A13 in FIG. 2).

Preferably, the annexin is capable of binding the phospholipid with a dissociation constant of about 10⁻⁶ M or less, about 10⁻⁷ M or less, about 10⁻⁸ M or less, or about 10⁻⁹ M or less, preferably about 10⁻¹⁰ M or less. Preferably, the annexin is capable of binding the phospholipid with a dissociation constant of from about 10⁻⁸ M to about 10⁻¹⁰ M, preferably from about 20⁻⁹ M to about 10⁻¹⁰ M, preferably about 50⁻¹⁰ M.

A conjugate according to the invention comprises a non-internalising annexin. Non-internalising annexins are described in Ungethum et. al. (2011) the Journal of Biological Chemistry 286, 3, 1903-1910 (which is incorporated herein by reference) and typically have one or more amino acids replaced in helices IA, ID, IIA, IID, IIIC, IIID and IVE and in the stretches connecting helices IC and ID, IIE and IIIA, IIIC and IIID, IIID and IIIE, and IVA and IVB. Preferably, one or more amino acids selected from polar amino acids His, Glu, Gln, Asp, Asn, Arg and Lys are replaced by non-polar amino acids. Suitably, the one or more amino acids are located at positions 16-29, 59-74, 88-102, 135-145, 156-169, 202-231, 259-266 and 305-317 of SEQ ID NO:1 for annexin A5, or the corresponding sequences for other annexins.

Preferably, one or more amino acids selected from Glu, Gln, Asp, Asn, Arg, Lys and His at positions 16-29, 59-74, 88-102, 135-145, 156-169, 202-231, 259-266 and 305-317 of SEQ ID NO:1 for annexin A5 or the corresponding sequences for other annexins are replaced by Gly, Ala, Val, Ile, Leu, Ser, Thr, Met, Pro, Phe, or Tyr.

A conjugate according to the invention preferably comprises a non-internalising annexin wherein at one or more, least 2, 3, 4, 5 or 6 of the polar amino acids are replaced by non-polar amino acids.

A conjugate according to the invention can comprise a non-internalising annexin having a cysteine residue at one or more amino acid positions selected from 1-15, 46-58, 86-87, 118-134, 170, 245-248 and 280-294 of annexin A5 and the corresponding stretches in other annexins, preferably at position 2.

Once bound to phospholipid, a conjugate according to the invention comprising a non-internalising annexin will remain on the cellular surface for a longer period of time compared to a wild-type annexin. The conjugate may provide improved and/or sustained masking of the immunosuppressive effects of PS exposed on the surface of cancer cells and the tumor vasculature, thus blocking a “checkpoint” utilised by the cancer, particularly a tumour and cells of the tumour microenvironment.

Suitably, the non-internalising annexin may have an impaired ability to form trimers. Suitably, the non-internalising annexin may have an impaired ability to form a two-dimensional network on the cellular surface. Preferably, the non-internalising annexin may have an impaired ability to form trimers and a two-dimensional network on the cellular surface.

A conjugate according to the invention comprises one or more immunostimulatory agents. Preferably, the immunostimulatory agent promotes an inflammatory response. The immunostimulatory agent can be selected from TNFα, IL1α, IL1β, IL2, IL4, IL6, IL8, IL10, IL12, IL15, IL17A, IFNγ, GM-CSF (CSF2), M-CSF (CSF1), G-CSF (CSF 3), an immunoglobulin, and fragments, monomers, multimers, variants, muteins, post-translationally modified versions thereof and mixtures thereof.

The immunostimulatory agent can be a fragment crystallizable (Fc) region of an immunoglobulin, monomer or fragment thereof. The immunoglobulin can be selected from IgG, IgM, IgA, and IgE, in particular their fragments. The immunoglobulin can be IgG. The IgG may be selected from IgG1, IgG2, IgG3 or IgG4, preferably IgG1 or IgG3, even more preferably IgG1. Conjugates according to the invention can comprise an Fc region of an immunoglobulin in the human or murine form, preferably human.

A conjugate according to the invention preferably comprises one or more human IgG1 Fc region, fragment, monomer, multimer, variant, mutein, post-translationally modified version thereof or mixture thereof.

A conjugate according to the invention can comprise an IgG Fc region with a modified amino acid sequence, or modified carbohydrates attached to the Fc region, to enhance complement dependent cytotoxicity (CDC), antibody dependent cell cytotoxicity (ADCC), and antibody dependent cellular phagocytosis (ADCP). For a review see Liu et. al. (2008) Immunological Reviews 222, 9-27 (which is incorporated herein by reference). For example, mutations of the IgG Fc region such as a double amino acid substitution Lys222Trp and Thr223Trp, or double substitution of Cys220Asp and Asp221Cys, or quadruple substitution of Cys220Asp and Asp221Cys and Lys222Trp and Thr223Trp, are some examples of mutations that enhance CDC. As another example, triple substitutions of Ser298Ala and Glu333Ala and Lys334Ala, or double amino acid substitution of Ser239Asp and Ile332Glu, enhance ADCC.

In this manner, a conjugate according to the invention can simultaneous shield PS from macrophage PS receptors and bind to macrophage Fcγ receptors. Such an interaction helps shift the polarization of tumour associated macrophages (TAMs) into the M1 phenotype and alter the cytokine balance within the tumour.

Modifications can also be undertaken to either enhance or reduce Fc binding to the neonatal Fc receptor (FcRn) in order to modify the conjugates' circulation times in vivo. For example, amino acid residues located in the interface between the IgG Fc constant domains Cγ2 and Cγ3 are involved in FcRn binding. As an example, modifications to one or more amino acids, such as Ile253, Ser254, Arg255, Lys288, Leu309, Ser415, His433, His435 and Tyr436 are predicted to reduce binding to human FcRn. Modifications to one or more amino acids such as Pro238, Thr256, Glu272, Val305, Thr307, His310, Gln311, Asp312, Lys317, Asp376, Ala378, Glu380, Glu382, Ser424 and Asn434 are predicted to enhance binding to human FcRn (see Table 4 of Martin et al. (2001) Molecular Cell 7, 867-877 [which is incorporated herein by reference]).

The immunostimulatory agent can be TNF-α. The term “TNF-α” is used according to its plain ordinary meaning in the art and refers to is a cell signaling protein (cytokine) involved in systemic inflammation and is one of the cytokines that make up the acute phase reaction. TNF-α includes modified proteins and homologs of the same. Human TNF-α may be a non-glycosylated soluble protein of 17 kDa and a length of 157 amino acids. Murine TNF-α is N-glycosylated. Homology with TNF-beta is approximately 30%. The 17 kDa form is produced by processing of a precursor protein of 233 amino acids. A transmembrane form of TNF-α of 26 kDa has also been described.

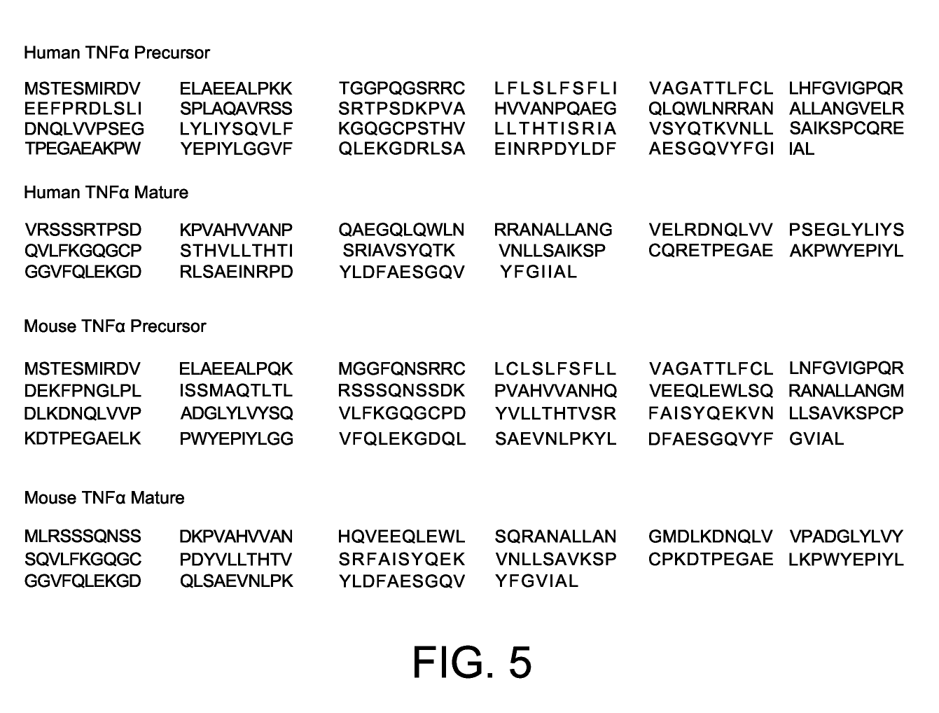

A conjugate according to the invention may comprises TNF-α in the soluble or transmembrane form, preferably the soluble form. Conjugates according to the invention can comprise TNF-α as a full protein, precursor or fragment thereof (as described in U.S. Pat. No. 7,892,558, which is incorporated herein by reference). Conjugates according to the invention can comprise TNF-α in the human or murine form, preferably human. TNF-α can be post-translationally modified. A conjugate according to the invention can comprise glycosylated or non-glycosylated TNF-α. Preferably, the conjugate according to the invention comprises SEQ ID NO: 14, 15, 16 or 17 (see FIG. 5).

A conjugate according to the invention can comprise TNF-α variants. Variants can be modified genetically (muteins) or chemically to be more or less toxic. For example, mutations Ala84 to Val and Val91 to Ala reduce the cytotoxic activity of the factor almost completely. Deletion of 7 N-terminal amino acids and the replacement of Pro8Ser9Asp10 by ArgLysArg yields a mutated factor with an approximately 10-fold enhancement in antitumor activity, increased receptor binding (L-M cell assay) and reduced toxicity. Other TNF-α variants are described in U.S. Pat. No. 7,446,174; U.S. Pat. No. 7,056,695; U.S. Pat. No. 7,118,750; U.S. Pat. No. 6,878,370; U.S. Pat. No. 7,642,340; patent applications US20020110868A1, US20070172449A1 and US20070207961A1, all of which are incorporated herein by reference. Variants can be modified chemically with small molecules or with artificial (e.g. poly-ethyleneglycol) or natural (e.g. carbohydrates) polymers. TNF-α variants may improve annexin trimerization and thereby enhance toxicity.

A conjugate according to the invention preferably comprises one or more human TNF-α, fragment, monomer, multimer, variant, mutein, post-translationally modified version thereof or mixture thereof.

In this manner, a conjugate according to the invention can simultaneously shield PS from macrophage PS receptors and promote an inflammatory response.

The conjugate of the present invention provides improved and/or sustained immunostimulatory effects.

A conjugate according to the invention can comprise a non-internalising annexin and a plurality of immunostimulatory agents, preferably two or three immunostimulatory agents.

The non-internalising annexin can be linked to the immunostimulatory agent via a linker. Moieties can be linked through conjugation of the N- or C-terminus of the annexin protein, through conjugation of the amines (lysine or arginine), sulfhydryls (e.g. cysteine), carboxyls (e.g. c-terminus), carbohydrates (e.g. oxidized sugars) or non-selectively (e.g. photoreactive crosslinking agents, formaldehyde, glutaraldehyde). Linkage can occur to amino acids found in nature or artificially constructed amino acids introduced into the sequence of the non-internalizing annexin or the immunostimulatory agent.

Linkers can be peptides, proteins, carbohydrates, nucleic acids, morpholinos (synthetic oligonucleotide analogues containing morpholino-phosphorodiimidate chains instead of deoxyribose-phosphodiester chains), peptide nucleic acids (synthetic oligonucleotide analogues containing N-aminoethyl-glycine chains instead of deoxyribose-phosphodiester chains, PNA).

The linker preferably comprises a peptide that promotes product stability and in vivo half-life. Examples of such linkers are Glycine-Serine linkers: (GGGGS)n; helix-forming peptide linkers: A(EAAAK)nA (Arai et al. 2001 Protein Engineering); a sequence of Pro, Ala and/or Ser amino acids: (PASn). Other peptide linkers are proteins or protein fragments that function as a spacer. In one example, “tags” can be used to link the annexin to the immunostimulatory agent. These tags can be a histidine tag (e.g. 6×His) or larger (e.g. c-myc, chitin binding protein (CBP), maltose binding protein (MBP), and glutathione-S-transferase (GST), human influenza hemagglutinin (HA) or the FLAG tag DYKDDDDK)). Linkers can be N-tails of other annexins, preferably an annexin selected from any of the annexins in FIG. 2.

Preferably, the linker can be a peptide linker comprising 1-50 amino acids, preferably about 1-10, 1-20, 1-30, 1-40, 1-50; about 20-30, 20-40, 20-50; about 30-40, 30-50; about 40-50 amino acids; preferably 1, 5, 10, 15, 20, 25, 30, 35, 40, 45, 50 amino acids.

Linkers can be non-cleavable or cleavable (e.g. cleavage using thiols, acids, bases, hydroxylamines or periodates). Linkers consisting of a crosslinking reagent can be homo-bi-functional (e.g. amine to amine) or hetero-bi-functional (e.g. amine to sulfhydryl). These crosslinkers can have lengths ranging from zero angstroms (e.g. formaldehyde), a defined length of, for example, 95.2 angstroms (SM(PEG)n NHS-PEG-Maleimide Crosslinkers from Life Technologies) and greater (e.g. glutaraldehyde). Linkers can be a thioether (e.g. succinimidyl-4-(N-maleimidomethyl) cyclohexane-1-carboxylate), known also by its name TAP (Tumour-Activated Prodrug) linker.

The non-internalising annexin can be linked to the immunostimulatory agent covalently or non-covalently. Linkers can be streptavidin/avidin and biotin combination, combination of complementary DNA and RNA oligonucleotides, complementary DNA and RNA analogs such as morpholinos (synthetic oligonucleotide analogues containing morpholino-phosphorodiimidate chains instead of deoxyribose-phosphodiester chains), peptide nucleic acids (synthetic oligonucleotide analogues containing N-aminoethyl-glycine chains instead of deoxyribose-phosphodiester chains, PNA) and aptamers (specifically binding oligonucleotides or oligopeptides), the antibody and hapten combination, and the receptor and ligand combination.

Preferably, the linker may be from 0 to about 200 angstroms in length; preferably from about 0-100; about 1-10, 1-20, 1-30, 1-40, 1-50, 1-60, 1-70, 1-80, 1-90 or 1-100 angstroms in length; about 10-20, 10-30, 10-40, 10-50, 10-60, 10-70, 10-80, 10-90 or 10-100 angstroms in length; about 20-30, 20-40, 20-50, 20-60, 20-70, 20-80, 20-90 or 20-100 angstroms in length; about 30-40, 30-50, 30-60, 30-70, 30-80, 30-90 or 30-100 angstroms in length; about 40-50, 40-60, 40-70, 40-80, 40-90 or 40-100 angstroms in length; about 50-60, 50-70, 50-80, 50-90 or 50-100 angstroms in length; about 60-70, 60-80, 60-90 or 60-100 angstroms in length; about 70-80, 70-90 or 70-100 angstroms in length; about 80-90 or 80-100 angstroms in length; or about 90-100 angstroms in length; preferably about 1, 5, 10, 15, 20, 25, 30, 35, 40, 45, 50, 55, 60, 65, 70, 75, 80, 85, 90, 95 or 100 angstroms in length.

Preferably, the linker may be about 17, about 24, about 32, about 39, about 53, or about 95 angstroms in length.

Preferably the non-internalising annexin may be linked to the immunostimulatory agent via the N- or C-terminus of the annexin, preferably via the N-terminus. Preferably the linkage is covalent.

A conjugate according to the invention can comprise a plurality of linkers. Where the conjugate comprises a plurality of immunostimulatory agents, one or more linkers can link each immunostimulatory agent to the annexin or to each other.

Phospholipid bilayers may further form liposomes or vesicles. Liposomes are bubble like structures formed from the lipid bilayers. Liposomes may contain small amounts of other molecules (e.g., immunostimulatory agents). Liposomes can vary in range of size from micrometers to tens of micrometers. Liposomes may comprise any of the phospholipids disclosed herein.

A conjugate according to the invention may include a liposome. In some embodiments, the annexin is conjugated to a liposome. In some embodiments, the liposome can include other molecules (e.g. immunostimulatory agents).

The invention relates to a polypeptide comprising a non-internalising annexin and an immunostimulatory agent as defined herein and a nucleotide sequence encoding such a polypeptide. Preferably, the polypeptide comprises a linker as defined herein.

The present invention provides conjugates for use in medical treatment. In particular, a conjugate of the invention can be used to treat cancer. The invention is particularly useful in treating cancer characterised by cells displaying extracellular PS, PI, PIP, PIP2 or PIP3; or by displaying a different quantity of extracellular PE compared to healthy, normal mammalian cells. Preferably, the cancer cells display greater than 20% of PE in the outer leaflet of the cellular membrane. Preferably, the cancer cells display greater than 30% of PE in the outer leaflet of the cellular membrane.

Preferably, the invention provides conjugates for treating tumour cells and cells of the tumour microenvironment. Cells of the tumour microenvironment include tumour vasculature and stromal cells (e.g. fibroblasts and immune cells) that support the tumour. Tumour cells and cells of the tumour microenvironment display PS, PI, PIP, PIP2 or PIP3 abnormally in their outer leaflet, preferably PS; or display a different quantity of extracellular PE compared to healthy, normal mammalian cells. Suitably, the tumour is selected from breast, triple negative breast, ovarian, prostate, castrate-resistant prostate, pancreatic, bladder, bone, head and neck, lung, liver, thyroid, esophageal, stomach, intestinal, brain, glioblastoma.

Healthy, normal mammalian cells can be distinguished from cancer cells based on the presence of phosphatidylserine (PS), phosphatidyl inositol (PI) or its phosphoinositide derivatives (PIP, PIP2, PIP3) in the outer leaflet of the membrane bilayer; or a different quantity of phosphatidyl ethanolamine (PE) in the outer leaflet of the membrane bilayer. PS, for example, can be detected on the extracellular surface of cells using radiolabelled or fluorescently labelled annexin A5 using flow cytometry of disaggregated cells, fluorescent microscopy of tissue sections, or in vivo imaging of PS exposed cells with SPECT imaging. Another technique is to add a conjugation agent to modify any PS that is exposed on the outer leaflet. Cells are then disrupted to quantify modified vs. unmodified PS. Unmodified PS would represent PS on the inner leaflet while modified PS would represent PS on the outer leaflet.

Methods of treating cancer involve administering a therapeutically effective amount of the conjugate according to the invention to an animal or patient in need of such treatment, optionally with one or more pharmaceutically acceptable excipient.

Suitably, the cancer can be treated by administering only a single therapeutic agent. For example, if the conjugate according to the invention is administered intravenously, the treatment can consist of only a single procedure for the patient.

In another embodiment, the invention provides a method of preparing a conjugate according to the invention comprising conjugating a non-internalising annexin having a cysteine residue at one or more amino acid positions selected from 1-15, 46-58, 86-87, 118-134, 170, 245-248 and 280-294 of annexin A5 and the corresponding stretches in other annexins, preferably at position 2, to an immunostimulatory agent; optionally via a linker as described herein.

The invention provides a method of preparing a fusion protein or nucleic acid as described herein using methods known in the art.

### Examples

### Non-Internalising Annexin A5 IgG Fc

Non-internalizing annexin A5 is manufactured in a microbial system, purified by chromatographic methods and then chemically conjugated at the N-terminal cysteine site to IgG Fc acquired commercially.

The gene construct encoding a polypeptide chain comprising a non-internalizing annexin A5 fused to Fc is transfected into microbial and mammalian expression systems.

### Non-Internalising Annexin A5 TNF-α

Non-internalizing annexin A5 is manufactured in a microbial system, purified by chromatographic methods and then chemically conjugated at the N-terminal cysteine site to TNF-α acquired commercially.

The gene construct encoding a polypeptide chain comprising a non-internalizing annexin A5 fused to TNF-α is transfected into microbial and mammalian expression systems.

### Product Characterisation

Conjugates are tested for degradation using SDS-PAGE, product aggregation using size exclusion chromatography (SEC), product identity and stability using mass spectrometry. These are standard techniques. Binding potency to PS is evaluated using assays described in the literature.

Non cell-based assay format: PS is dissolved in n-hexane to a concentration of 50 μg/mL and applied to a 96-well plate in 100 μL aliquots. After evaporation of the solvent in a fume hood, the plates are blocked with a blocking agent and then incubated with serial dilutions of annexin A5 (AnxA5) conjugates for 2 hours at 25° C. before being rinsed with wash buffer (Tris buffered saline with 0.1% Tween 20) and incubated with an appropriate secondary antibody conjugated to horseradish peroxidase for 30 minutes at 25° C. (goat anti-mouse IgG for AnxA5-Fc; anti-TNFα and then goat anti-mouse IgG for AnxA-TNFα). Upon further washing, the plates are developed with TMB substrate and read at 450 nm wavelength.

Cell-based assay format: PS exposure is induced on the surface of cultured cells, without causing apoptosis, by treating cells either with 200 μM H₂O₂ in serum-free media for 1 hour at 37° C. or 20 pM docetaxel in media for 24 hours at 37° C. Cells are washed with PBS, incubated with AnxA5 conjugates in serum-free media for 1 hour at 25° C. before being washed again and then fixed with 4% (v/v) paraformaldehyde in PBS for 15 minutes. Binding of AnxA5 or the conjugates to the induced cells is compared with untreated cells using an anti-Annexin A5 antibody (e.g. Abcam cat. # ab14196) and a suitable secondary antibody conjugated to a fluorescent probe. The fixed cells can also be counterstained with DAPI to detect DNA in the nuclei.

Toxicity study: Using female BALB/c mice, groups of mice (n=3) receive 0, 0.3, 1, 3 and 10 mg/kg of AnxA5 conjugates on Day 1. Physical examination and survivability (at pre-treatment (−1), Day 1, Day 7); Body weights (on Days −14, −7, −1, 1, 7); Clinical observations (twice daily on days −7 to 7 plus pre-dose and 1, 6 h post-dose); Food and water consumption (on Day −7 to 7); Gross pathology and organ weights (including heart, liver, kidneys, GI tract, brain, lungs, etc.) are recorded and a maximum tolerated dose (MTD) identified. Harvested tissues are preserved in formalin for histopathology work.

PK study: Serum samples from 6 timepoints (3 mice/timepoint) are analysed using a sandwich ELISA capture assay. The studies will be conducted with IP administration, standard practice for tox studies in mice.

### Preclinical Studies in a Syngeneic Breast Cancer Model

The ability of AnxA5 conjugates to target and cause shrinkage of a xenografted tumor generated by an isogenic cultured breast cancer cell line (4T1) is determined. BALB/c mice bearing tumors are treated IV with candidate AnxA5 conjugates when the tumor burden reaches its pre-determined half-maximal value. Pharmacological distribution of the conjugates is determined at time of injection and at time of sacrifice. Tumor growth and response to treatment are monitored longitudinally while tumor histology and immune cell composition are determined upon sacrifice. These studies determine that an aggressive breast malignancy can be targeted and diminished in size using conjugates of the invention.

Experimental Design: Mouse breast cancer cells (4T1) are injected into the mammary fat pad of female BALB/c mice and allowed to grow to a half-maximal tumor volume. At that time the mice are randomized into four distinct groups (n=18/group) and treated intravenously with a vehicle control (PBS), unconjugated non-internalizing annexin A5, non-internalizing annexin A5 conjugated to IgG Fc (Anx A5-Fc), or non-internalizing annexin A5 conjugated to TNFα (AnxA5-TNFα). Mice are monitored on alternate days (weight, tumor dimension and whole animal imaging) until maximal disease burden is achieved in the vehicle control or treatment groups. When maximal disease burden is achieved the animals are sacrificed and processed for further analysis including a final size determination of the tumor, quantitation of the annexin A5 or its conjugates in the tissues and blood, and characterization of the immune cell infiltration into the tumor microenvironment post-treatment.

The 18 mice receiving Anx A5-Fc, the 18 receiving AnxA5-TNFα and the 18 receiving AnxA5 are further sub-divided into 3 groups (n=6) receiving either a low, medium or high dosage determined based on the MTD of the conjugates determined in the Toxicity study.

The study is then repeated using the optimal dosage for each of the constructs: non-internalizing annexin A5, Anx A5-Fc, Anx A5-TNFα or vehicle control (PBS). Each construct is tested again in groups of 18 mice in order to obtain statistically significant results.

### Preclinical Studies in a Breast Cancer Induction Model

The ability of conjugates according to the invention to target and cause tumor shrinkage in a genetic breast cancer model initiated by the polyoma middle T antigen which is driven by a mouse mammary tumor virus promoter (MMTV-PyMT) is determined. Tumor bearing mice are treated intravenously with candidate AnxA5 conjugates when the tumor burden reaches its pre-determined half-maximal value. Pharmacological distribution of the conjugates is determined at time of injection and at time of sacrifice. Tumor growth and response to treatment are monitored longitudinally while tumor histology and immune cell composition are determined upon sacrifice. Lung metastasis that occurs in this model allows the treatment of both primary and secondary disease to be assessed. Disease progression and disease burden can be diminished using conjugates of the invention.

Experimental Design: Female BALB/c MMTV-PyMT mice are allowed to progress to half-maximal tumor volume. When that tumor burden is reached, mice are selected to participate in four distinct groups and treated intravenously with a vehicle control (PBS), unconjugated non-internalizing annexin A5, non-internalizing annexin A5 conjugated to IgG Fc (Anx A5-Fc), or non-internalizing annexin A5 conjugated to TNFα (AnxA5-TNFα). Mice are monitored on alternate days (weight, tumor dimension) until maximal disease burden is achieved in the vehicle control or treatment groups. When maximal disease burden is achieved the animals are sacrificed and processed for further analysis including a final size determination of the tumor, quantitation of the annexin A5 or its conjugates in the tissues and blood, and characterization of the immune cell infiltration into the tumor microenvironment post-treatment.

18 mice receiving Anx A5-Fc, 18 receiving AnxA5-TNFα and 18 receiving AnxA5 are further sub-divided into 3 groups (n=6) receiving either a low, medium or high dosage determined based on the MTD of the conjugates.

The study is then repeated using the optimal dosage for each of the constructs: non-internalizing annexin A5, Anx A5-Fc, Anx A5-TNFα or vehicle control (PBS). Each construct is tested again in groups of 18 mice in order to obtain statistically significant results.

1. A conjugate comprising a non-internalising annexin and an immunostimulatory agent, wherein the non-internalising annexin is capable of binding to at least one phospholipid.

2. The conjugate according to claim 1, wherein the phospholipid is selected from phosphatidylserine (PS), phosphatidyl ethanolamine (PE), phosphatidyl inositol (PI) and phosphoinositide derivatives selected from PIP, PIP2 and PIP3.

3. The conjugate according to claim 2, wherein the phospholipid is PS.

4. The conjugate according to claim 1, wherein the non-internalising annexin is annexin A5.

5. The conjugate according to claim 1, wherein one or more amino acids selected from polar amino acids His, Glu, Gln, Asp, Asn, Arg and Lys in the helices IA, ID, IIA, IID, IIIC, IIID and IVE and in the stretches connecting helices IC and ID, IIE and IIIA, IIIC and IIID, IIID and IIIE, and IVA and IVB of wild type annexin are replaced by non-polar amino acids.

6. The conjugate according to claim 1, wherein the non-internalising annexin comprises SEQ ID NO:1 for annexin A5 or the corresponding sequences for other annexins wherein the one or more replaced amino acids are located at positions 16-29, 59-74, 88-102, 135-145, 156-169, 202-231, 259-266 and 305-317 of SEQ ID NO:1 for annexin A5, or the corresponding sequences for other annexins.

7. The conjugate according to claim 1, wherein the non-internalising annexin comprises SEQ ID NO:1 for annexin A5 or the corresponding sequences for other annexins, wherein one or more amino acids selected from Glu, Gln, Asp, Asn, Arg, Lys and His at positions 16-29, 59-74, 88-102, 135-145, 156-169, 202-231, 259-266 and 305-317, or the corresponding sequences for other annexins, are replaced by Gly, Ala, Val, Ile, Leu, Ser, Thr, Met, Pro, Phe, or Tyr.

8. The conjugate according to claim 5, wherein at least two of said polar amino acids are replaced by non-polar amino acids.

9. The conjugate according to claim 1, wherein the annexin binds to the phospholipid with a dissociation constant of about 10⁻⁶ M or less.

10. The conjugate according to claim 1, wherein the immunostimulatory agent promotes an inflammatory response.

11. The conjugate according to claim 1, wherein the immunostimulatory agent is selected from a nanoparticle, TNFα, IL1α, IL1β, IL2, IL4, IL6, IL8, IL10, IL12, IL15, IL17A, IFNγ, GM-CSF (CSF2), M-CSF (CSF1), G-CSF (CSF 3), an immunoglobulin, and fragments, monomers, multimers, variants, muteins, post-translationally modified versions thereof and mixtures thereof.

12. The conjugate according to claim 11, wherein the immunostimulatory agent is a fragment crystallizable (Fc) region of an immunoglobulin, monomer or fragment thereof.

13. The conjugate according to claim 11, wherein the immunoglobulin is IgG.

14. The conjugate according to claim 11, wherein the immunostimulatory agent is TNF-α.

15. The conjugate according to claim 1, wherein the non-internalising annexin is linked to the immunostimulatory agent via a linker.

16. The conjugate according to claim 15, wherein the non-internalising annexin is linked to the immunostimulatory agent via the N- or C-terminus.

17. The conjugate according to claim 1, wherein the non-internalising annexin is conjugated to a liposome.

18. The conjugate according to claim 1 comprising a plurality of immunostimulatory agents and optionally a plurality of linkers.

19. The conjugate according to claim 18, wherein the immunostimulatory agent is selected from a nanoparticle, TNFα, IL1α, IL1β, IL2, IL4, IL6, IL8, IL10, IL12, IL15, IL17A, IFNγ, GM-CSF (CSF2), M-CSF (CSF1), G-CSF (CSF 3), an immunoglobulin, and fragments, monomers, multimers, variants, muteins, post-translationally modified versions thereof and mixtures thereof.

20. A polypeptide comprising a conjugate according to claim 1.

21. A nucleotide sequence encoding a polypeptide according to claim 20.

22. (canceled)

23. (canceled)

24. (canceled)

25. (canceled)

26. (canceled)

27. (canceled)

28. A method of treating cancer comprising administering to a patient in need thereof an effective amount of a conjugate comprising a non-internalising annexin and an immunostimulatory agent, wherein the non-internalising annexin is capable of binding to at least one phospholipid.

29. The method according to claim 28, wherein the phospholipid is selected from phosphatidylserine (PS), phosphatidyl ethanolamine (PE), phosphatidyl inositol (PI) and phosphoinositide derivatives selected from PIP, PIP2 and PIP3.

30. The method according to claim 28, wherein the phospholipid is PS.

31. The method according to claim 28, wherein the non-internalising annexin is annexin A5.

32. The method according to claim 28, wherein one or more amino acids selected from polar amino acids His, Glu, Gln, Asp, Asn, Arg and Lys in the helices IA, ID, IIA, IID, IIIC, IIID and IVE and in the stretches connecting helices IC and ID, IIE and IIIA, IIIC and IIID, IIID and IIIE, and IVA and IVB of wild type annexin are replaced by non-polar amino acids.

33. The method according to claim 28, wherein the annexin comprises SEQ ID NO:1 for annexin A5 or the corresponding sequences for other annexins and wherein the one or more replaced amino acids are located at positions 16-29, 59-74, 88-102, 135-145, 156-169, 202-231, 259-266 and 305-317 of SEQ ID NO:1 for annexin A5, or the corresponding sequences for other annexins.

34. The method according to claim 33, wherein the non-internalising annexin comprises SEQ ID NO:1 for annexin A5 or the corresponding sequences for other annexins, wherein one or more amino acids selected from Glu, Gln, Asp, Asn, Arg, Lys and His at positions 16-29, 59-74, 88-102, 135-145, 156-169, 202-231, 259-266 and 305-317, or the corresponding sequences for other annexins, are replaced by Gly, Ala, Val, Ile, Leu, Ser, Thr, Met, Pro, Phe, or Tyr.

35. The method according to claim 32, wherein at least two of said polar amino acids are replaced by non-polar amino acids.

36. The method according to claim 28, wherein the annexin binds to the phospholipid with a dissociation constant of about 10⁻⁶ M or less.

37. The method according to claim 28, wherein the immunostimulatory agent promotes an inflammatory response.

38. The method according to claim 28, wherein the immunostimulatory agent is selected from TNFα, IL1α, IL1β, IL2, IL4, IL6, IL8, IL10, IL12, IL15, IL17A, IFNγ, GM-CSF (CSF2), M-CSF (CSF1), G-CSF (CSF 3), an immunoglobulin, and fragments, monomers, multimers, variants, muteins, post-translationally modified versions thereof and mixtures thereof.

39. The method according to claim 28, wherein the immunostimulatory agent is a fragment crystallizable (Fc) region of an immunoglobulin, monomer or fragment thereof.

40. The method according to claim 39, wherein the immunoglobulin is IgG.

41. The method according to claim 28, wherein the immunostimulatory agent is TNF-α.

42. The method according to claim 28, wherein the non-internalising annexin is linked to the immunostimulatory agent via a linker.

43. The method according to claim 42, wherein the non-internalising annexin is linked to the immunostimulatory agent via the N- or C-terminus.

44. The method according to claim 28, wherein the conjugate comprises a plurality of immunostimulatory agents and optionally a plurality of linkers.

45. The method according to claim 44, wherein the immunostimulatory agent is selected from a nanoparticle, TNFα, IL1α, IL1β, IL2, IL4, IL6, IL8, IL10, IL12, IL15, IL17A, IFNγ, GM-CSF (CSF2), M-CSF (CSF1), G-CSF (CSF 3), an immunoglobulin, and fragments, monomers, multimers, variants, muteins, post-translationally modified versions thereof and mixtures thereof.

46. The method according to claim 28, wherein a cancer cells display extracellular PS, PI or PI derivatives selected from PIP, PIP2 and PIP3; or displays a different quantity of extracellular PE compared to a healthy, normal mammalian cell.

47. The method according to claim 28, wherein the conjugate is capable of blocking the immunosuppressive effect of PS on the surface of cancer cells and causes immunostimulation.

48. The method according to claim 28, wherein the cancer is a solid tumour.

49. The method according to claim 48, wherein the tumour is selected from breast, triple negative breast, ovarian, prostate, castrate-resistant prostate, pancreatic, bladder, bone, head and neck, lung, liver, thyroid, esophageal, stomach, intestinal, brain, glioblastoma.
